(19) United States
(12) Patent Application Publication
(10) Pub. No.: US 2026/0287599 A1
(43) Pub. Date: Sep. 24, 2026

(54) GLYCINE RECEPTORS IN WHITE BLOOD CELLS AS BIOMARKERS FOR PAIN

(71) Applicants: Texas Tech University System, Lubbock, TX (US); University of Pittsburgh-Of the Commonwealth System of Higher Education, Pittsburgh, PA (US)

(72) Inventors: Huanyu Dou, El Paso, TX (US); Yan Xu, Wesford, PA (US)

(73) Assignees: Texas Tech University System, Lubbock, TX (US); University of Pittsburgh-Of the Commonwealth System of Higher Education, Pittsburgh, PA (US)

(21) Appl. No.: 19/168,386

(22) PCT Filed: Mar. 23, 2024
(86) PCT No.: PCT/US2024/021248

§ 371 Date: Sep. 24, 2025

Related U.S. Application Data
(60) Provisional application No. 63/454,565, filed on Mar. 24, 2023.

(51) Int. Cl. G01N 33/68; G16H 10/40; G16H 50/30

(52) U.S. Cl. CPC G01N 33/6896; G16H 10/40; G16H 50/30; G01N 2333/705; G01N 2800/2842; G01N 2800/52

(57) ABSTRACT

Embodiments of the present disclosure pertain to methods of assessing pain in a subject by receiving one or more measured levels of glycine receptors of the subject; and correlating differentially expressed levels of the glycine receptors to pain. The methods of the present disclosure may also include a step of implementing a treatment decision based on the assessment. In some embodiments, the methods of the present disclosure may be repeated after implementing the treatment decision. Additional embodiments of the present disclosure pertain to systems for assessing pain in a subject. In some embodiments, the systems include computer-readable storage mediums with program codes that implement the methods of the present disclosure.

### CROSS-REFERENCE TO RELATED APPLICATIONS

This application claims priority to U.S. Provisional Patent Application No. 63/454,565, filed on Mar. 24, 2023. The entirety of the aforementioned patent application is incorporated herein by reference.

### BACKGROUND

Current methods of assessing pain in patients have numerous limitations. Embodiments of the present disclosure aim to address the aforementioned limitations.

### SUMMARY

In some embodiments, the present disclosure pertains to methods of assessing pain in a subject. In some embodiments, the methods of the present disclosure include: receiving one or more measured levels of glycine receptors of the subject; and correlating differentially expressed levels of the one or more glycine receptors to pain. In some embodiments, the methods of the present disclosure also include a step of implementing a treatment decision based on the assessment. In some embodiments, the treatment decision includes monitoring the course of the pain, implementing a pain prevention regimen, implementing a pain treatment regimen, and/or implementing a pain management regimen. In some embodiments, the methods of the present disclosure may be repeated after implementing the treatment decision.

Additional embodiments of the present disclosure pertain to systems for assessing pain in a subject. In some embodiments, the system includes computer-readable storage mediums having a program code embodied therewith. In some embodiments, the program code includes programming instructions for: receiving one or more measured glycine receptor levels of the subject; and correlating differentially expressed levels of the one or more glycine receptors to pain. In some embodiments, the systems of the present disclosure also include programming instructions for recommending a treatment decision based on the assessment (e.g., monitoring the course of the pain, implementing a pain prevention regimen, implementing a pain treatment regimen, and/or implementing a pain management regimen).

### BRIEF DESCRIPTION OF THE DRAWINGS

FIG. 1A illustrates a method of assessing pain in a subject in accordance with various embodiments of the present disclosure.

FIG. 1B illustrates a system for assessing pain in a subject in accordance with various embodiments of the present disclosure.

FIGS. 2A-2C illustrate the expression of α3 glycine receptor (GlyR) in human and mouse white blood cells. FIGS. 2A-2B show immunofluorescent staining of α3GlyR (green) expressed in mouse (FIG. 2A) and human (FIG. 2B) white blood cells (WBCs) and co-localized with CD11b+ monocytes (light grey) and CD3+ lymphocytes (red), as shown in the merged images, where cell nuclei were counterstained by DAPI (blue). FIG. 2C shows flow cytometry analysis showing distinctly different α3GlyR expression between WBCs from control mice (red) and from mice experiencing inflammatory pain induced by Complete Freund's adjuvant (CFA) injection after 24 hours (cyan). The subset of α3GlyR gated on α3GlyR side scatter histogram shows a significant reduction of α3GlyR counts in the CFA-pain mice (9.74%, cyan) compared to the control mice (31.8%, red).

FIGS. 3A-3F show GlyRs expression in Human WBCs. FIG. 3A shows immunostaining for α3GlyR (green), α1GlyR (red) and DAPI (blue) exhibited α1 and α3GlyRs expressed on the surface of human WBCs. FIG. 3B shows dual labeling WB assay with housekeeping control GAPDH (red) indicates a ~50 kD α3GlyR (green) in human WBCs. Representative plots and quantitative flow cytometry analysis detected that α3GlyR is highly expressed in CD11b+, CD68+ and Ly6C+ subsets (FIGS. 3C and 3E) when compared to lymphocytes (FIGS. 3D and 3F).

FIGS. 4A-4D show WBCs and differentiated bone marrow-derived macrophages (BMMs) from C57BL/6 mice. Double labeling with antibodies against α1 and α3 GlyR was performed on WBCs (FIG. 4A) and BMMs (FIG. 4B). Images exhibited α1 and α3 GlyR expression in the surface of cell membrane α1GlyR (red in the middle panels of FIGS. 4A-4B, arrow heads in the right panels of FIGS. 4A-4B) or α3GlyR (green in the left panels of FIGS. 4A-4B, arrows in the right panels of FIGS. 4A-4B). FIGS. 4C and 4D show the validation of antibodies by the WB assay. HEK293T/17 cells (HEK) without or with a1 and α3 GlyR transfection (α1GlyR+ HEK and α3GlyR+ HEK) were labeled with anti-α1GlyR (NovusBio Cat #NB300-113) and anti-α3GlyR (Antibodies, Inc Cat #75-417) antibodies, indicating the specificity of anti-α3GlyR for α3GlyR.

FIGS. 5A-5H show that CFA-induced reduction of α3GlyR+ WBCs correlates with inflammatory pain. The WBCs were isolated from the normal C67BL/6 mice (control) and mice having CFA-induced inflammation pain at 24 hrs as a peak pain condition. Flow cytometry analysis of α3GlyR+ WBCs was paired with CD3+, CD8+, CD45+, and Ly6C+ labeling. Representative plots showed the decreases of WBCs with co-expression of α3GlyR+CD3+ (FIG. 5A), α3GlyR+CD8+ (FIG. 5B), α3GlyR+CD45+ (FIG. 5C), and α3GlyR+Ly6C+ (FIG. 5D) in a CFA-induced pain model. FIG. 5E shows the subset of α3GlyR gated on α3GlyR/counts histogram, which shows different α3GlyR expression between WBCs from control mice (red, n=3), and from CFA-induced pain model (cyan, n=5). FIG. 5F shows significant reduction of α3GlyR+ subsets from CFA-injected mice, indicating a negative correlation. FIG. 5G shows the percentage of α3GlyR+ WBCs (green) normalized by DAPI nuclei (blue), which confirmed the negative correlation in CFA mice (FIG. 5H, p<0.01).

FIGS. 6A-6B show partial separation of pain from inflammation, which suggests pain specificity of α3GlyR reduction in WBCs. FIG. 6A shows WB of α3GlyRs from mice 15 d after CFA injection (lanes 1-2) when inflammation has subsided; control mice (lanes 3-4); and mice 24 h after CFA injection when both pain and inflammation were present (lanes 5-6). The housekeeping GAPDH serves as a loading reference. FIG. 6B shows quantitative analyses of α3GlyR expression in FIG. 6A, as normalized by GAPDH and naïve control. Note that even after inflammation has subsided, CFA pain still suppresses α3GlyR expression in the WBCs (lanes 1-2), with only a minor recovery from the severe pain 24 h after CFA injection (lanes 5-6). A tear in the gel on the right edge artificially shifted up the α3GlyR band in lane 6.

FIGS. 7A-7B establish that pain-free inflammation does not reduce the expression of α3GlyRs in WBCs. An in vitro LPS-induced inflammation model was designed to determine whether inflammation itself without pain regulates the expression of α3GlyR in WBCs. WBCs were isolated from C57BL/6 mice and half of the cultures were treated with LPS (300 ng/ml) for 24 hours. The untreated WBCs from the same batch served as controls. FIG. 7A shows dual labeling of α3GlyR (green) and DAPI (blue), which shows that α3GlyR expressions in WBC cultures were the same with or without LPS-induced inflammation. The results from flow cytometry (FIG. 7B) confirmed that LPS-induced inflammation in WBC cultures did not downregulate α3GlyR+ cell populations (cyan: control; red: with LPS-induced inflammation).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

A strong need exists for a fast and noninvasive test to evaluate pain in patients and quantify pain sensation. In particular, pain is the most common reason for physician consultation in most developed countries. Furthermore, pain is a major symptom of many medical conditions, which can interfere with a person's quality of life and general functioning. However, no objective biomarkers are currently available for non-invasive determination and evaluation of pain, such as chronic pain involving inflammation or neuropathy. Numerous embodiments of the present disclosure aim to address the aforementioned limitations.

### Methods of Assessing Pain

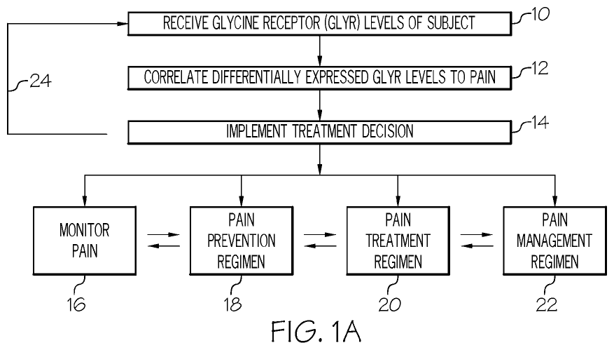

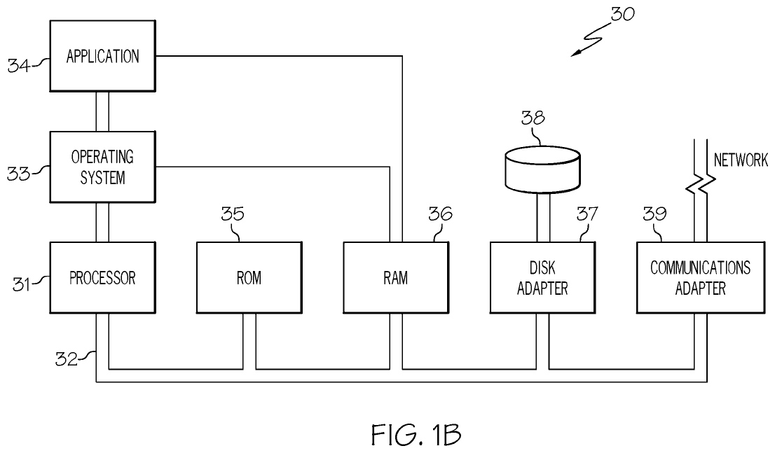

In some embodiments, the present disclosure pertains to methods of assessing pain in a subject. In some embodiments illustrated in FIG. 1A, the methods of the present disclosure include: receiving one or more measured levels of glycine receptors of the subject (step 10); and correlating differentially expressed levels of the one or more glycine receptors to pain (step 12). In some embodiments, the methods of the present disclosure also include a step of implementing a treatment decision based on the assessment (step 14). In some embodiments, the treatment decision includes monitoring the course of the pain (step 16), implementing a pain prevention regimen (step 18), implementing a pain treatment regimen (step 20), and/or implementing a pain management regimen (step 22). In some embodiments, the methods of the present disclosure may be repeated after implementing the treatment decision (step 24).

Additional embodiments of the present disclosure pertain to systems for assessing pain in a subject. In some embodiments, the system includes computer-readable storage mediums having a program code embodied therewith. In some embodiments, the program code includes programming instructions for: (1) receiving one or more measured glycine receptor levels of the subject; and (2) correlating differentially expressed levels of the one or more glycine receptors to pain. In some embodiments, the systems of the present disclosure also includes programming instructions for recommending a treatment decision based on the assessment (e.g., monitoring the course of the pain, implementing a pain prevention regimen, implementing a pain treatment regimen, and/or implementing a pain management regimen).

As set forth in more detail herein, the methods and systems of the present disclosure can have numerous embodiments and applications.

### Assessment of Pain

In some embodiments, pain may generally refer to a protective evolutionary function that involves unpleasant sensory and emotional experiences associated with, or resembling that associated with, actual or potential tissue damage. In some embodiments, pain may also generally refer to a distressing feeling often caused by intense or damaging stimuli.

The methods and systems of the present disclosure can be utilized to assess various types of pain. According to the International Association for the Study of Pain, pain is defined as “an unpleasant sensory and emotional experience associated with, or resembling that associated with, actual or potential tissue damage, and is expanded upon by the addition of six key Notes and the etymology of the word pain for further valuable context” (reference: PAIN 161(9):p 1976-1982, September 2020.|DOI: 10.1097/j. pain.0000000000001939). For instance, in some embodiments, the pain includes, without limitation, nociceptive pain, inflammatory pain, neuropathic pain, psychogenic pain, chemotherapy-induced pain, chronic versions thereof, acute versions thereof, or combinations thereof. Based on the length of temporal measures, pain may be classified as acute or chronic pain. For instance, in some embodiments, the pain includes chronic pain involving inflammation or neuropathy.

In some embodiments, the pain includes psychogenic pain when physical causes are ruled out by diagnoses. For instance, headaches, muscle pains, back pain, stomach pain are non-limiting examples of psychogenic pain. In some embodiments, the pain may be classified as neuropathic pain due to injuries, malfunctions, or abnormalities of nerves. Neuropathic pains may also be sub-divided into peripheral and central neuropathic pain dependent on the location of nerve injuries or malfunctions.

In some embodiments, the pain includes chemotherapy-induced pain. In some embodiments, the chemotherapy-induced pain includes chemotherapy-induced neuropathic pain, such as chemotherapy-induced nerve injury pain.

Pain may be presented in various forms. For instance, in some embodiments, pain may be presented as a form of hypersensitivity or allodynia to thermal or mechanical stimulations.

### Glycine Receptor Levels

The methods and systems of the present disclosure may measure and correlate various glycine receptor levels to pain. For instance, in some embodiments, the measured glycine receptor levels include, without limitation, measured levels of alpha 1 subtype of the glycine receptor (α1 GlyR), alpha 2 subtype of the glycine receptor (α2GlyR), alpha 3 subtype of the glycine receptor (α3GlyR), alpha 4 subtype of the glycine receptor (α4GlyR), beta subtype of the glycine receptor, or combinations thereof. In some embodiments, alpha subtypes of glycine receptors can form functional receptors from a single subtype or from mixing with the beta subtype. However, beta subtype receptors alone typically do not form functional receptors. In some embodiments, the measured glycine receptor levels include an alpha 3 containing subtype of the glycine receptor (e.g., α3GlyR and/or α3βGlyR). In some embodiments, the measured glycine receptor levels include an alpha 1 containing subtype of the glycine receptor (e.g., α1GlyR and/or α1βGlyR).

Differentially expressed levels of glycine receptors may have various representations. For instance, in some embodiments, the differentially expressed levels of the glycine receptors represent elevated levels of the glycine receptors, depressed levels of the glycine receptors, or combinations thereof. In some embodiments, the differentially expressed levels of the glycine receptors represent elevated or depressed levels of the glycine receptors relative to normal levels of the glycine receptors.

In some embodiments, differentially expressed levels of glycine receptors represent elevated levels of the glycine receptors. In some embodiments, elevated levels of glycine receptors represent glycine receptor levels that are at least 10% higher than the average glycine receptor levels of subjects that are not suffering from pain. In some embodiments, elevated levels of glycine receptors represent glycine receptor levels that are at least 15% higher than the average glycine receptor levels of subjects that are not suffering from pain. In some embodiments, elevated levels of glycine receptors represent glycine receptor levels that are at least 20% higher than the average glycine receptor levels of subjects that are not suffering from pain. In some embodiments, elevated levels of glycine receptors represent glycine receptor levels that are at least 25% higher than the average glycine receptor levels of subjects that are not suffering from pain. In some embodiments, elevated levels of glycine receptors represent glycine receptor levels that are at least 50% higher than the average glycine receptor levels of subjects that are not suffering from pain.

In some embodiments, differentially expressed levels of glycine receptors represent depressed levels of the glycine receptors. In some embodiments, depressed levels of glycine receptors represent glycine receptor levels that are at least 10% lower than the average glycine receptor levels of subjects that are not suffering from pain. In some embodiments, depressed levels of glycine receptors represent glycine receptor levels that are at least 15% lower than the average glycine receptor levels of subjects that are not suffering from pain. In some embodiments, depressed levels of glycine receptors represent glycine receptor levels that are at least 20% lower than the average glycine receptor levels of subjects that are not suffering from pain. In some embodiments, depressed levels of glycine receptors represent glycine receptor levels that are at least 25% lower than the average glycine receptor levels of subjects that are not suffering from pain. In some embodiments, depressed levels of glycine receptors represent glycine receptor levels that are at least 50% lower than the average glycine receptor levels of subjects that are not suffering from pain.

Differentially expressed levels of glycine receptors may have various forms. For instance, in some embodiments, the differentially expressed levels of the glycine receptors include differentially expressed mRNA levels of the glycine receptors, differentially expressed protein levels of the glycine receptors, or combinations thereof. In some embodiments, the differentially expressed levels of the glycine receptors include differentially expressed mRNA levels of the glycine receptors. In some embodiments, the differentially expressed levels of the glycine receptors include differentially expressed protein levels of the glycine receptors.

### Samples

In some embodiments, the measured glycine receptor levels may be obtained from a biological sample of a subject. In some embodiments, the biological sample includes at least one of a tissue sample, body fluid, blood sample, or combinations thereof. In some embodiments, the biological sample includes a blood sample.

Measured glycine receptor levels may be obtained from various cells of a subject. For instance, in some embodiments, the measured glycine receptor levels are obtained from non-neuronal cells of the subject. In some embodiments, the non-neuronal cells include immune cells. In some embodiments, the immune cells include, without limitation, white blood cells, neutrophils, eosinophils, basophils, mast cells, monocytes, macrophages, peripheral blood mononuclear cells (PBMCs), bone marrow-derived macrophages (BMMs), dendritic cells, natural killer cells, lymphocytes, B cells, T cells, and combinations thereof. In some embodiments, the immune cells include white blood cells. In some embodiments, the immune cells include bone marrow-derived macrophages (BMMs).

In some embodiments, the methods and systems of the present disclosure also include a step of or programming instructions for measuring glycine receptor levels from a biological sample of a subject. In some embodiments, the methods and systems of the present disclosure also include a step of or programming instructions for obtaining the biological sample from the subject.

### Subjects

The methods and systems of the present disclosure may be utilized to assess pain in various subjects. For instance, in some embodiments, the subject is a human being. In some embodiments, the subject is a non-human mammal. In some embodiments, the non-human mammal includes, without limitation, a horse, a rabbit, a mouse, a rat, a pig, a sheep, a cow, a dog, or a cat. In some embodiments, the non-human mammal is a domestic animal, such as a dog or a cat.

In some embodiments, the subject is suffering from pain. In some embodiments, the subject is vulnerable to pain.

In some embodiments, the subject is unable to express feelings of pain. For instance, in some embodiments, the subject may be a human being suffering from one or more conditions that impair speech. In some embodiments, such conditions may include, without limitation, Alzheimer's disease, dementia, Parkinson's disease, or combinations thereof.

### Assessment of Pain in Subjects

The methods and systems of the present disclosure may assess pain in subjects in various manners. For instance, in some embodiments, the assessment includes correlating differentially expressed levels of glycine receptors to at least one of diagnosis of pain, assessment of pain, quantification of pain, progression of pain, or combinations thereof. In some embodiments, the assessment includes correlating differentially expressed levels of glycine receptors to diagnosis of pain.

In some embodiments, the assessment includes correlating elevated levels of glycine receptors to low intensity pain, and correlating decreased levels of glycine receptors to high intensity pain. In some embodiments, the assessment includes correlating elevated levels of glycine receptors to high intensity pain, and correlating decreased levels of glycine receptors to low intensity pain. In some embodiments, assessment of pain levels uses survey instruments composed of questionaries to evaluate subjective unpleasant experiences of physical, emotional, and psychological pain. In some other embodiments, the instruments include subjective reporting scales from 0 for no discomfort to 10 for intolerable discomfort or pain. In yet other embodiments, the scale can be from 0 to 100. In some other embodiments, the same survey instruments are repeatedly used before and after medical interventions to ameliorate pain. In some embodiments where a 0-10 scale is used, 1-3 may be considered low intensity pain, 4-6 may be considered mid intensity pain, and 7-10 may be considered high intensity pain.

### Pain Assessment Modes

Assessment of pain in subjects may occur in various modes. For instance, in some embodiments, the assessment occurs manually. In some embodiments, the assessment occurs in real-time. In some embodiments, the assessment occurs continuously.

In some embodiments, the assessment occurs automatically through the utilization of an algorithm. In some embodiments, the systems of the present disclosure include the algorithm. In some embodiments, the algorithm is an L1-regularized logistic regression algorithm. In some embodiments, the algorithm is a machine learning algorithm trained on the glycine receptors. In some embodiments, the machine learning algorithm includes supervised learning algorithms. In some embodiments, the supervised learning algorithms include nearest neighbor algorithms, naïve-Bayes algorithms, decision tree algorithms, linear regression algorithms, support vector machines, neural networks, convolutional neural networks, ensembles (e.g., random forests and gradient boosted decision trees), or combinations thereof.

Machine learning algorithms may be trained in various manners. For instance, in some embodiments, the training includes: (1) feeding a first set of measured glycine receptor levels into a machine learning algorithm, where the first set of measured glycine receptor levels are from one or more subjects that are suffering from pain; (2) feeding a second set of measured glycine receptor levels into the machine learning algorithm, where the second set of measured glycine receptor levels are from one or more subjects that are not suffering from pain; and (3) training the machine learning algorithm to assess pain in the subject by comparing the first set of measured glycine receptor levels with the second set of glycine receptor levels.

### Implementing or Recommending a Treatment Decision

In some embodiments, the methods of the present disclosure also include a step of implementing a treatment decision based on the assessment of pain in a subject. In some embodiments, the systems of the present disclosure also include programming instructions for recommending a treatment decision based on the assessment of pain in a subject. In some embodiments, the treatment decision includes monitoring the course of the pain, implementing a pain prevention regimen, implementing a pain treatment regimen, implementing a pain management regimen, or combinations thereof.

In some embodiments, the treatment decision includes implementing a pain treatment regimen. In some embodiments, the pain treatment regimen includes administering a therapeutic agent to the subject. In some embodiments, the pain treatment regimen includes a modified version of a pre-existing pain treatment regimen.

In some embodiments, the methods of the present disclosure are repeated after implementing the treatment decision. In some embodiments, the method is utilized to continuously assess the efficacy of the treatment decision.

### Applications

The methods and systems of the present disclosure provide numerous advantages and applications. In particular, no objective biomarkers are currently available for non-invasive determination and evaluation of pain, such as neuropathic pain and inflammation pain. As such, the methods and systems of the present disclosure can find clinical applications in diagnosis, assessment, quantification, and treatment efficacy evaluation of pain, such as acute and chronic pain. For instance, in some embodiments, the methods and systems of the present disclosure may be utilized to evaluate the efficacy of drugs in treating or preventing pain. In some embodiments, the methods and systems of the present disclosure may be utilized to evaluate the efficacy of different dosages of the drugs in treating or preventing pain.

### Systems

The systems of the present disclosure may have various architectures and forms. For instance, in some embodiments, the systems of the present disclosure are in the form of a web-based program, an application-based program, or combinations thereof. In some embodiments, the systems of the present disclosure include an algorithm, such as a machine-learning algorithm trained on the glycine receptors. Suitable algorithms were described supra and are incorporated herein by reference.

The systems of the present disclosure can include various types of computer-readable storage mediums. For instance, in some embodiments, the computer-readable storage medium can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, or combinations thereof. A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, or combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions for the systems of the present disclosure can be downloaded to respective computing/processing devices from a computer-readable storage medium or to an external computer or external storage device via a network, such as the Internet, a local area network (LAN), a wide area network (WAN) and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages.

In some embodiments, the computer-readable program instructions may execute entirely on the user's computer, partly on the user's computer, as a stand-alone software package, partly on the user's computer and partly on a remote computer or entirely on the remote computer or server. In the latter scenario, the remote computer may be connected in some embodiments to the user's computer through any type of network, including a LAN or a WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure for assessing pain as discussed herein may be implemented using a system illustrated in FIG. 1B. Referring now to FIG. 1B, FIG. 1B illustrates an embodiment of the present disclosure of the hardware configuration of a system 30 which is representative of a hardware environment for practicing various embodiments of the present disclosure.

System 30 has a processor 31 connected to various other components by system bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for assessing pain as discussed in the present disclosure, such as in connection with FIGS. 1A, 2A-2C, 3A-3F, 4A-4D, 5A-5H, 6A-6B, and 7A-7B.

Referring again to FIG. 1B, read-only memory (“ROM”) 35 is connected to system bus 32 and includes a basic input/output system (“BIOS”) that controls certain basic functions of system 30. Random access memory (“RAM”) 36 and disk adapter 37 are also connected to system bus 32. It should be noted that software components including operating system 33 and application 34 may be loaded into RAM 36, which may be system's 30 main memory for execution. Disk adapter 37 may be an integrated drive electronics (“IDE”) adapter that communicates with a disk unit 38 (e.g., a disk drive). It is noted that the program for assessing pain, as discussed in the present disclosure, such as in connection with FIGS. 1A, 2A-2C, 3A-3F, 4A-4D, 5A-5H, 6A-6B, 7A-7B may reside in disk unit 38 or in application 34.

System 30 may further include a communications adapter 39 connected to system bus 32. Communications adapter 39 interconnects system bus 32 with an outside network (e.g., wide area network) to communicate with other devices.

Aspects of the present invention are described herein with reference to flowchart illustrations and/or block diagrams of methods, apparatus (systems), and systems according to embodiments of the invention. It will be understood that each block of the flowchart illustrations and/or block diagrams and combinations of blocks in the flowchart illustrations and/or block diagrams can be implemented by computer-readable program instructions.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks. The computer-readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and systems according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. The Changes of Membrane Glycine Receptor Alpha 3 Expression During Pain, Inflammation and Treatment

The glycine receptor (GlyR) is the receptor of the amino acid neurotransmitter glycine. GlyR is an ionotropic receptor that produces its effects through chloride current. It is one of the most widely distributed inhibitory receptors in the central nervous system and has important roles in a variety of physiological processes, especially in mediating inhibitory neurotransmission that is associated to the pain conditions.

Pain arising from inflammation, tissue damage, or nerve injury changes the inhibitory-excitatory balance allowing normally innocuous tactile sensory neurons to access nociceptive neurons. Activation of GlyRs reduces excitatory neurotransmission and causes net inhibition. GlyRs are critical for regulating nociception. In fact, decreases of GlyRs have been found to be associated with hyperalgesia in several experimental pain models, indicating significant roles for GlyRs (α1GlyR, α3GlyR) in maintaining normal inhibitory-excitatory balance.

Applicants discovered that α1GlyR and α3GlyR are highly expressed on human and mouse white blood cells (WBCs). In particular, the expression of α3GlyR was significantly reduced in mouse inflammation pain models.

### Example 1.1. GlyR α3 Expression in Blood White Cells (WBCs)

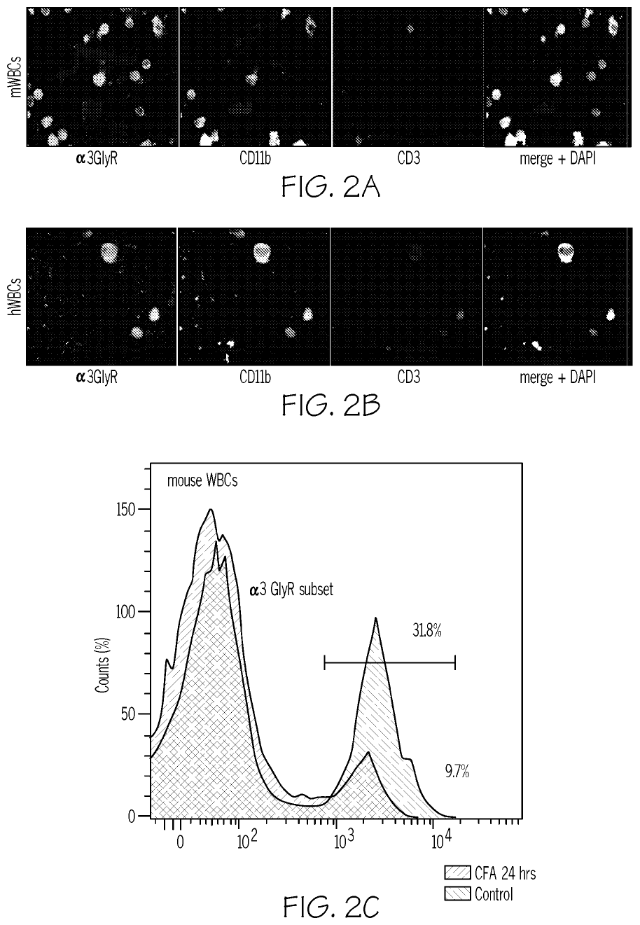

Immunohistochemistry staining shows GlyRα3 expression in mouse (FIG. 2A) and human (FIG. 2B) CD11b+ monocytes. The overall α3GlyR+ cell counts are significantly reduced in mice 24 h after Complete Freund's adjuvant (CFA)-induced pain compared to the naïve controls (FIG. 2C). The chronic pain conditions modify the expression of GlyRα3 in WBCs, which in turn will modify the pain inhibitory signals.

### Example 1.2. GlyRs α1 and α3 Expression in Human WBCs

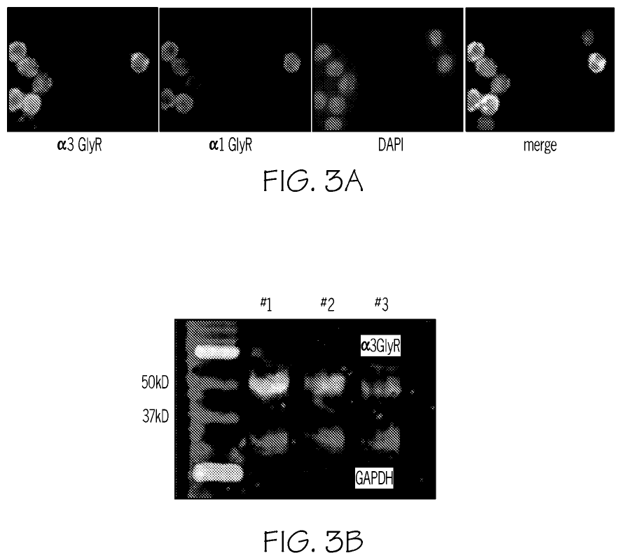

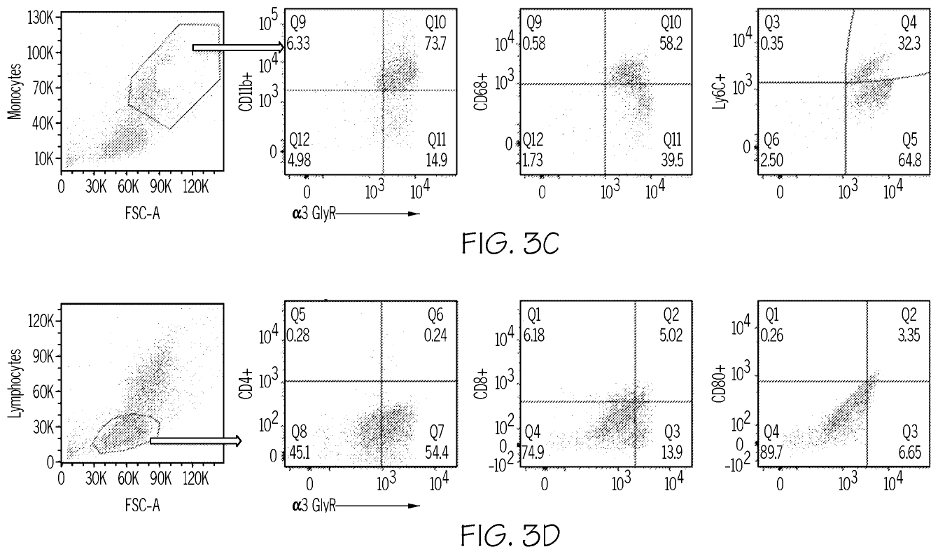

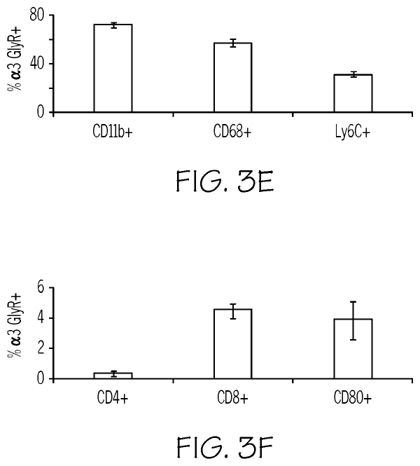

Human WBCs were used to determine the expression of GlyRs by immunofluorescence staining, Western Blot (WB), and flow cytometry assays. α3GlyR and α1GlyR fluorescence staining showed GlyR α1 (red), and a3 (green) are expressed on the surface of WBCs (FIG. 3A). WB assay (FIG. 3B) illustrated a ~50 kD α3GlyR protein band (green) and 37 kD GAPDH (red). Flow cytometry analysis of subsets of WBCs showed that α3GlyR was largely expressed in CD11b+, CD68+, and Ly6C+ cells (FIGS. 3C and 3E). In contrast, fewer lymphocytes were α3GlyR+ (FIGS. 3D and 3F).

### Example 1.3. GlyRs α1 and α3 Expression in Mouse WBCS and Bone Marrow-Derived Macrophages (BMMs)

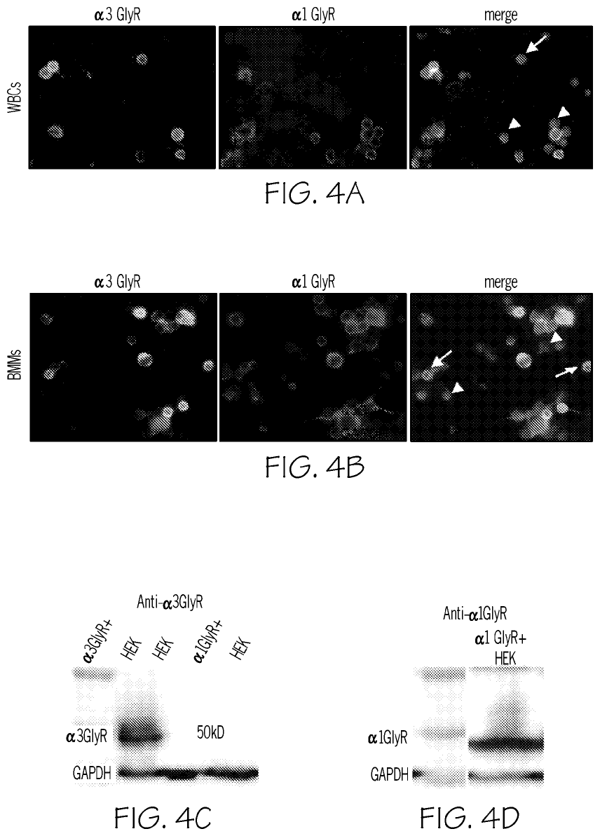

The WBCs and bone marrow-derived macrophages (BMMs) were obtained from C57BL/6 mice. BMMs were differentiated for 12 days in vitro. Immunostaining showed that al (red) and α3 (green) GlyRs are expressed on the surface of WBCs and BMMs (FIGS. 4A and 4B). A large proportion of WBCs and BMMs co-express α1 and a3 GlyRs. Fewer WBCs expressed only α1GlyR+ (red in the left panels of FIGS. 4A-4B, arrow heads in the right panels of FIGS. 4A-4B) or α3GlyR+ (green in the left panels of FIGS. 4A-4B, arrows in the right panels of FIGS. 4A-4B). These data indicate that α1 and α3 GlyR expressions differ in the subsets of WBCs and BMMs. The specificity of antibodies against α3GlyR and α1GlyR (FIGS. 4C and 4D) were validated and used for the immunostaining, flow cytometry and WB assays.

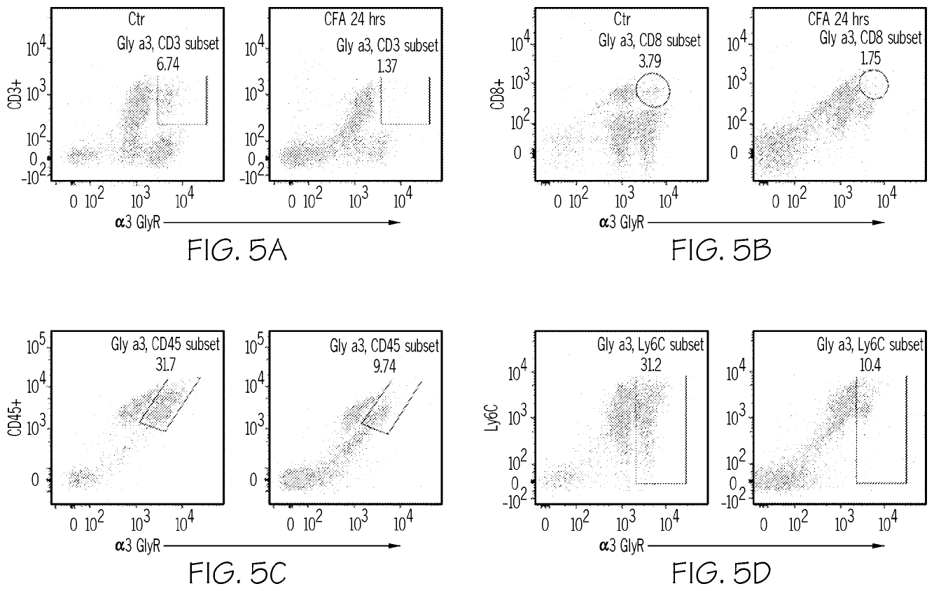

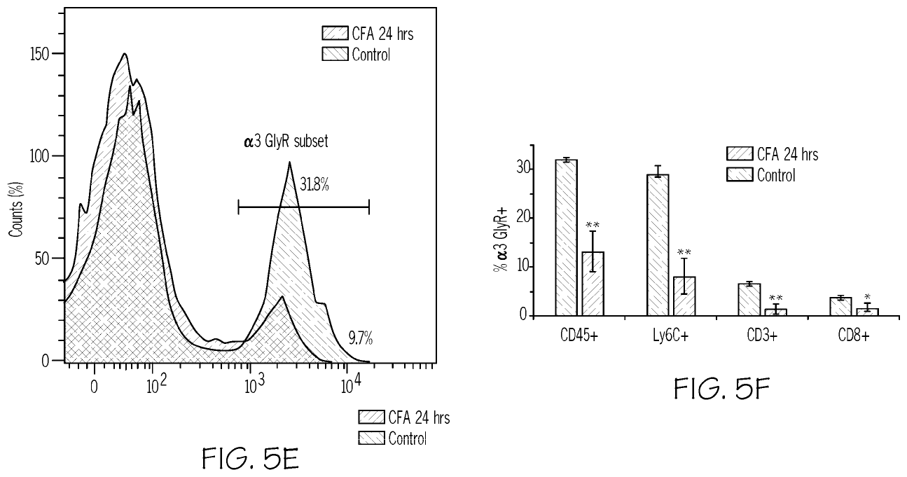

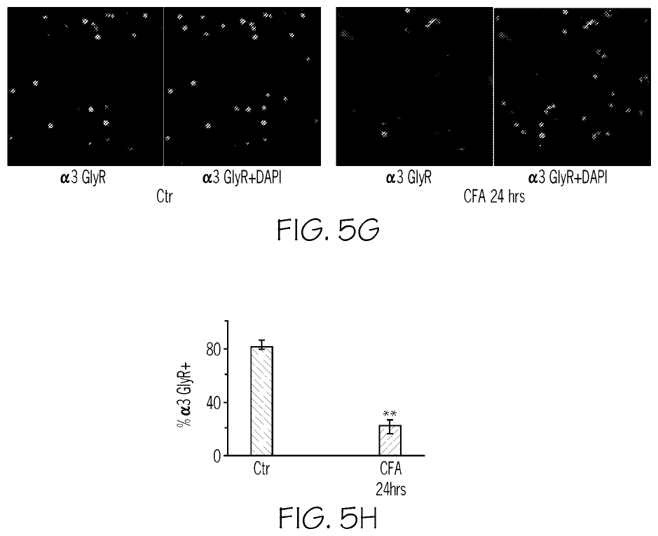

Example 1.4. A Reduction of α3GlyR+ WBCs Correlates with Inflammatory Pain Applicants further investigated the changes in α3GlyR+ WBCs induced by inflammatory pain. C67BL/6 mice (n=5) were injected with Complete Freund Adjuvant (CFA, 10 μl) into the left hind paw. Naïve mice (n=3) served as the control. The blood was collected from the control and CFA mice at 24 hrs when the painful condition peaked. WBCs were isolated for flow cytometry and immunostaining assays. Representative flow cytometry plots displayed the WBCs gated on α3GlyR+ and paired to CD3+, CD8+, CD45+, and Ly6C+. A reduction of α3GlyR+ subsets was observed in CFA-induced inflammatory pain mice (FIGS. 5A-5D). Notably, up to 31.8% of the control WBCs were α3GlyR+, but it was significantly decreased to 9.7% in CFA-induced pain mice at 24 hrs (FIG. 5E). Further, the expression of α3GlyR was significantly reduced in CD45+, LY6C+, CD3+ and CD8+ subsets (FIG. 5F, **p<0.01; *p<0.05). Dual immunostaining of α3GlyR (FIG. 5G, green) and DAPI nuclei (FIG. 5G, blue) showed a large reduction of α3GlyR+ WBCs in the CFA-induced inflammation pain mice as compared to the naïve control. Quantitative analysis confirmed the decrease of α3GlyR+ in the CFA-induced inflammation pain mice (FIG. 5H, p<0.01).

### Example 1.5. Reduction of α3GlyR+ in WBCs is Specific to Pain

Applicants carried out two sets of experiments to partially separate pain from inflammation and determined if the downregulation of α3GlyR resulted from pain or inflammation (although the two are not always separable in vivo). In the first set, Applicants waited for the CFA-induced inflammation to subside. It has been shown that resolution of CFA-induced inflammation occurs within 1-2 weeks.

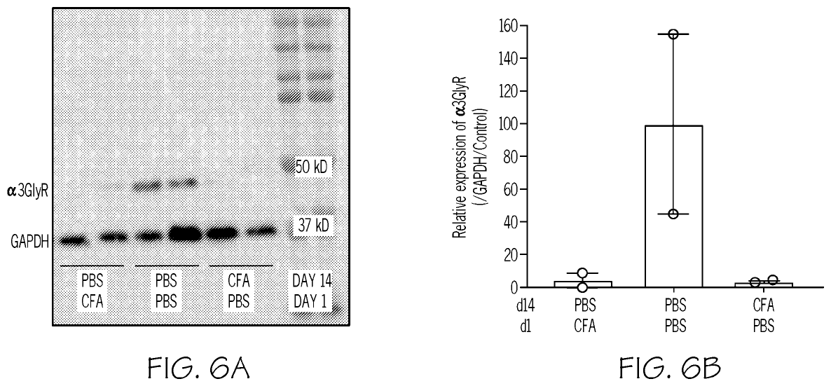

However, CFA-induced pain lasts for 6 weeks or longer. FIG. 6A shows α3GlyR WB from blood of three animal groups. Group 1 received a 5 μL CFA injection into the hind paw on Day 1 and a 5 μL phosphate buffered saline (PBS) injection on Day 14 and were sacrificed for blood collection on Day 15 to serve as the delayed CFA pain group with reduced inflammation. Group 2 received 5 μL PBS injections on Day 1 and Day 14 and were sacrificed on Day 15 to serve as the negative (no pain) control. Group 3 received a 5 μL PBS injection on Day 1 and 5 μL CFA injection on Day 14 and were sacrificed on Day 15 to serve as positive control of pain+inflammation. As can be seen in the quantitative analysis in FIG. 6B, even after inflammation has subsided while pain is still present, the α3GlyR level is still depressed (left bar) compared to the negative control (middle bar), and only slightly recovered (though not significantly) compared to the severe pain group (right bar). Therefore, without being bound by theory, Applicants believe that the degree of decrease in α3 GlyR 24 h after CFA injection (right bar) is predominantly caused by pain instead of inflammation.

In the second set of experiments, Applicants used an in vitro experimental condition to create inflammation without pain to determine if inflammation alone can cause downregulation of α3GlyR in WBCs. Lipopolysaccharide (LPS) is widely recognized as a potent activator to induce inflammation, which is always associated with pain in vivo. To isolate inflammation from pain, Applicants created an in vitro model of inflammation by treating WBCs in culture with LPS (300 ng/ml). WBCs were isolated from C57BL/6 mice and cultured with LPS for 24 h. The untreated WBCs from the same batch served as control. Immunostaining was performed to determine whether inflammation alone can downregulate α3GlyR expression in WBCs.

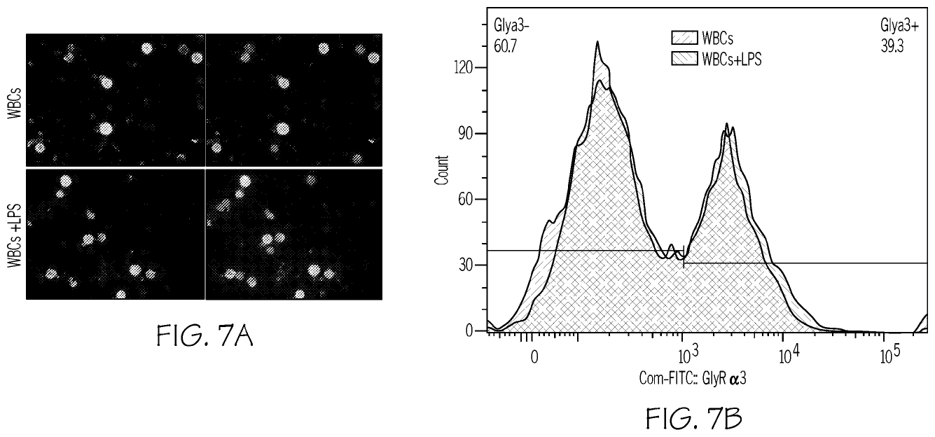

Dual-label images showed no measurable changes in α3GlyR expression (FIG. 7A, green) in the LPS-treated cultures compared to the control, based on normalization by DAPI+ cell counts (FIG. 7A, blue). Flow cytometry quantification of α3GlyR+ WBCs (FIG. 7B) showed that the level of α3GlyR expression was essentially the same in the WBCs with and without LPS-induced inflammation. These results suggest that the reduction of α3GlyR expression observed in the in vivo inflammatory pain models can be attributed largely to the pain condition, lending additional support to further validate the use of α3GlyR expression patterns as potential blood biomarkers for pain.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of assessing pain in a subject, said method comprising:
receiving one or more measured levels of glycine receptors of the subject, wherein the one or more glycine receptor levels is obtained from non-neuronal cells of the subject; and
correlating differentially expressed levels of the one or more glycine receptors to pain.

2. The method of claim 1, wherein the pain is selected from the group consisting of nociceptive pain, inflammatory pain, neuropathic pain, psychogenic pain, chemotherapy-induced pain, chronic versions thereof, acute versions thereof, or combinations thereof.

3. The method of claim 1, wherein the one or more glycine receptors is selected from the group consisting of alpha 1 subtype of the glycine receptor (α1GlyR), alpha 2 subtype of the glycine receptor (α2GlyR), alpha 3 subtype of the glycine receptor (α3GlyR), alpha 4 subtype of the glycine receptor (α4GlyR), beta subtype of the glycine receptor, or combinations thereof.

4. The method of claim 1, wherein the one or more glycine receptors comprises an alpha 3 containing subtype of the glycine receptor.

5. The method of claim 1, wherein the one or more glycine receptors comprises an alpha 1 containing subtype of the glycine receptor.

6. The method of claim 1, wherein the differentially expressed levels of the one or more glycine receptors represent elevated levels of the one or more glycine receptors.

7. The method of claim 1, wherein the differentially expressed levels of the one or more glycine receptors represent depressed levels of the one or more glycine receptors.

8. The method of claim 1, wherein the non-neuronal cells comprise immune cells.

9. The method of claim 8, wherein the immune cells are selected from the group consisting of white blood cells, neutrophils, eosinophils, basophils, mast cells, monocytes, macrophages, peripheral blood mononuclear cells (PBMCs), bone marrow-derived macrophages (BMMs), dendritic cells, natural killer cells, lymphocytes, B cells, T cells, and combinations thereof.

10. The method of claim 8, wherein the immune cells comprise white blood cells.

11. The method of claim 8, wherein the immune cells comprise bone marrow-derived macrophages (BMMs).

12. The method of claim 1, wherein the method further comprises a step of measuring the one or more glycine receptor levels from a biological sample of the subject.

13. The method of claim 1, wherein the subject is a human being.

14. The method of claim 1, wherein the subject is a non-human mammal.

15. The method of claim 1, wherein the assessment comprises correlating differentially expressed levels of the one or more glycine receptors to at least one of diagnosis of pain, assessment of pain, quantification of pain, progression of pain, or combinations thereof.

16. The method of claim 1, wherein the assessment comprises correlating differentially expressed levels of the one or more glycine receptors to diagnosis of pain.

17. The method of claim 1, wherein the assessment comprises correlating elevated levels of the one or more glycine receptors to low intensity pain, and correlating decreased levels of the one or more glycine receptors to high intensity pain.

18. The method of claim 1, further comprising a step of implementing a treatment decision based on the assessment.

19. The method of claim 18, wherein the decision comprises monitoring the course of the pain, implementing a pain prevention regimen, implementing a pain treatment regimen, implementing a pain management regimen, or combinations thereof.

20. The method of claim 18, wherein the treatment decision comprises implementing a pain treatment regimen, wherein the pain treatment regimen comprises administering a therapeutic agent to the subject.

21. The method of claim 18, wherein the method is repeated after implementing the treatment decision.

22. The method of claim 1, wherein the method is utilized to evaluate the efficacy of one or more drugs in treating or preventing pain.

23. The method of claim 22, wherein the method is utilized to evaluate the efficacy of different dosages of the one or more drugs in treating or preventing pain.

24. A system for assessing pain in a subject, wherein the system comprises one or more computer-readable storage mediums having a program code embodied therewith, wherein the program code comprises programming instructions for:
receiving one or more measured levels of glycine receptors of the subject; and
correlating differentially expressed levels of the one or more glycine receptors to pain.

25. The system of claim 24, wherein the system comprises programming instructions for correlating differentially expressed levels of the one or more glycine receptors to at least one of diagnosis of pain, assessment of pain, quantification of pain, progression of pain, or combinations thereof.

26. The system of claim 24, wherein the system comprises programming instructions for correlating elevated levels of the one or more glycine receptors to low intensity pain, and correlating decreased levels of the one or more glycine receptors to high intensity pain.

27. The system of claim 24, wherein the system comprises an algorithm.

28. The system of claim 27, wherein the algorithm is a machine learning algorithm trained on the one or more glycine receptors.

29. The system of claim 24, wherein the system further comprises programming instructions for recommending a treatment decision based on the assessment.

30. The system of claim 29, wherein the treatment decision comprises monitoring the course of the pain, implementing a pain prevention regimen, implementing a pain treatment regimen, implementing a pain management regimen, or combinations thereof.

31. The system of claim 24, wherein the one or more glycine receptor levels is obtained from non-neuronal cells of the subject.
